(19) United States
(12) Patent Application Publication
(10) Pub. No.: US 2009/0099592 A1
(43) Pub. Date: Apr. 16, 2009

(54) Detachable Interlock Systems and Methods of Use

(75) Inventors: Marcia Buiser, Marlborough, MA (US); Christopher Nardone, N. Chelmsford, MA (US); Robert Rioux, Ashland, MA (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Boston Scientific Scimed, Inc.

(21) Appl. No.: 12/250,977

(22) Filed: Oct. 14, 2008

Related U.S. Application Data
(60) Provisional application No. 60/979,979, filed on Oct. 15, 2007.

(51) Int. Cl. A61M 29/00; A61F 5/04

(52) U.S. Cl. 606/200; 606/53

(57) ABSTRACT

Described herein are various implant delivery systems and methods of use. The systems can include an implantable medical device mated to a pusher wire via a detachable link. The user can detach the detachable link by rotating the pusher wire. For example, the detachable link can include first and second interlocking members, where the first interlocking member including a male mating surface and the second interlocking member including a female mating surface. The first and second body members can be mechanically interlocked, for example, with a pin and a slot.

### BACKGROUND

This application claims priority to Provisional Application Ser. No. 60/979,979 entitled “DETACHABLE INTERLOCK SYSTEMS AND METHODS OF USE” filed Oct. 15, 2007, which is incorporated herein by reference.

The endovascular treatment of a variety of maladies throughout the body is an increasingly more important form of therapy. One such procedure uses embolizing coils to occlude a target site by posing a physical barrier to blood flow and/or by promoting thrombus formation at the site. Such treatments can be useful where it is desired to reduce vascularization, including treatments for aneurisms and cancer.

Coils have typically been placed at a desired site within the vasculature using a catheter and a pusher. As a first step, a flexible, small diameter catheter can be guided to the target site through the use of guidewires or by flow-directed means such as balloons placed at the distal end of the catheter. Once the site has been reached, the catheter lumen is cleared by removing the guidewire (if a guidewire has been used), and the coil is placed into the proximal open end of the catheter and advanced through the catheter with a pusher. Pushers are wires having a distal end that is adapted to engage and push the coil through the catheter lumen as the pusher is advanced through the catheter. When the coil reaches the distal end of the catheter, it is discharged from the catheter by the pusher into the vascular site.

Several techniques have been developed to enable more accurate placement of coils within a vessel. In one technique the coil is bonded via a metal-to-metal joint to the distal end of the pusher. The pusher and coil are made of dissimilar metals. The coil-carrying pusher is advanced through the catheter to the site and a small electrical current is passed through the pusher-coil assembly. The current causes the joint between the pusher and the coil to be severed via electrolysis. The pusher may then be retracted leaving the detached coil at an exact position within the vessel. In addition to enabling accurate coil placement, the electric current may facilitate thrombus formation at the coil site. A perceived disadvantage of this method is that the electrolytic release of the coil requires a period of time so that rapid detachment of the coil from the pusher does not occur.

Another technique for detaching an embolic coil uses a mechanical connection between the coil and the pusher. For example, one such device uses interlocking clasps which are secured to each other by a control wire that extends the length of the catheter. Retraction of the control wire uncouples the coil from the pusher. While mechanical connections between coils and pusher wires provide for quick detachment, such detachable coils require additional control mechanisms (i.e., control wires) to deploy the coil.

Accordingly, while conventional systems provide effective coil delivery, further improvements that reduce the chance of premature deployment or jamming would be beneficial.

### SUMMARY

Described herein are systems and methods for delivering an implantable device to a target site using a detachable link. The described systems overcome the drawbacks of conventional delivery devices and provide a more robust coil delivery system that allows a user to control when and where an implant is detached from a control wire. In addition, the systems can reduce the chance of premature detachment and/or jamming. In one such embodiment, the system can include a detachable link which can be unlocked by rotating a control wire.

In one embodiment, the implant delivery system includes an implantable medical device mated to a pusher wire via a detachable link comprising first and second interlocking members. The first interlocking member has a male mating surface and the second interlocking member has a female mating surface. Mechanically engagement of the male and female surfaces can be achieved via a pin and a slot.

In one aspect, the slot is positioned on an inner wall of the female mating surface and the pin is positioned on an outer portion of the male mating surface. At least a portion of the slot can extend in a non-longitudinal direction. For example, the slot can extend transversely around a portion of a circumference of the female mating surface. The slot can include multiple segments extending in different directions. For example, the slot can have a first elongate portion and a second elongate portion extending at an angle with respect to the first elongate portion.

In one aspect, the male mating surface is closed-ended. Alternatively, the male surface can have an outer wall defining an inner region. In another aspect, the female mating surface includes a wall defining a recessed area. The female mating surface can include an open distal end, a recessed area, and a closed proximal end.

The depth of the slot can be less than the thickness of the wall of the female mating surface. Alternatively, the slot can extend through the wall. In one aspect, the system can include multiple pins and multiple slots.

In a further aspect, the first and second body members mechanically interlock without frictionally mating. For example, the slot can have a width greater than a width of the pin to allow the pin to freely slide in the slot. In addition, the shape and size of the female mating surface can be configured to allow the male mating surface to engage/disengage with minimal friction.

In another aspect, the female mating surface can include a pin and the male mating surface can include a slot. For example, the pin can extend from the inner wall of the female mating surface and the slot can be positioned in an outer region of the male mating surface.

In another embodiment, an implant delivery system is provided herein. The system comprises an implantable medical device mated to a pusher wire via a detachable link comprising an elongate body member including first and second interlocking members. The first interlocking member includes an outer surface having at least one protrusion and the second interlocking member including an wall defining a receiving area for receiving at least a portion of the first interlocking member. The wall further includes a recess adapted to receive the protrusion when the first and second interlocking members are engaged.

In yet another embodiment, a method of delivering a detachable implant is provided. The method can include providing an embolic coil mated to a pusher wire via a detachable link comprising first and second interlocking members. The first interlocking member includes an outer surface having at least one protrusion, and the second interlocking member including an wall defining a receiving area for receiving at least a portion of the first interlocking member. The wall can further including a slot for receiving the protrusion. A surgeon can actuate the pusher wire to move the embolic coil through a catheter. Once the coil is in position, the user can rotate the pusher wire to detach the pusher wire from the embolic coil.

In a further aspect, the step of rotating results in the first interlocking member rotating relative to the second interlocking member. For example, the force required to rotate the embolic coil and the first interlocking member can be greater than the force required to rotate the first interlocking member relative to the second interlocking member.

In another aspect, a distal portion of the catheter can be configured to facilitate detachment. For example, the distal portion can include a cross-sectional shape adapted to facilitate detaching the embolic coil. The cross-sectional shape can have at least one smaller cross-sectional width than the first and/or second interlocking member. In another aspect, the distal portion of the catheter has surface features adapted to grip one of the interlocking members.

In another aspect, the method described herein can include determining the location of the detachable link relative to the catheter and/or an anatomical features. For example, a user can determine the location of the detachable link and/or coil inside the catheter using tactile feedback and/or a visualization technique.

### BRIEF DESCRIPTION OF THE DRAWINGS

The accompanying drawings, which are incorporated in and constitute a part of this specification, illustrate illustrative embodiments of the invention and, together with the description, serve to explain the principles of the invention.

FIG. 1A illustrates a side view of an embodiment of the implant delivery system described herein;

FIG. 1B illustrates a perspective view of the system of FIG. 1A in a detached configuration;

FIG. 2A illustrates a perspective view of another embodiment of the system described herein;

FIG. 2B illustrates a perspective view of the system of FIG. 2A in a detached configuration;

FIG. 3A illustrates a front (proximal) view of an interlocking member used with the system described herein;

FIG. 3B illustrates a perspective view of another embodiment of the interlocking member of FIG. 3A;

FIG. 4A illustrates a side view of an interlocking member used with the system described herein;

FIG. 4B illustrates a side view of another embodiment of the interlocking member of FIG. 4A;

FIG. 4C illustrates a side view of yet another embodiment of the interlocking member of FIG. 4A;

FIG. 5A illustrates a perspective view of another embodiment of an interlocking member described herein;

FIG. 5B illustrates a perspective view of yet another embodiment of an interlocking member described herein; and

FIG. 6 illustrates a perspective view of yet another embodiment of an interlocking member described herein.

### DETAILED DESCRIPTION

Disclosed herein are methods and systems for delivering an implantable device to a target site, particularly, a detachable, implantable device. The detachable, implantable device can be mated to a pusher wire via a detachable link that comprises first and second interlocking members. Discussed below are a variety of detachable links which include features that inhibit unwanted detachment during delivery through a catheter while allowing a user to control detachment when the implantable device reaches a target area. In one embodiment, the interlocking members allow the user to control detachment of implantable device by rotating the pusher wire.

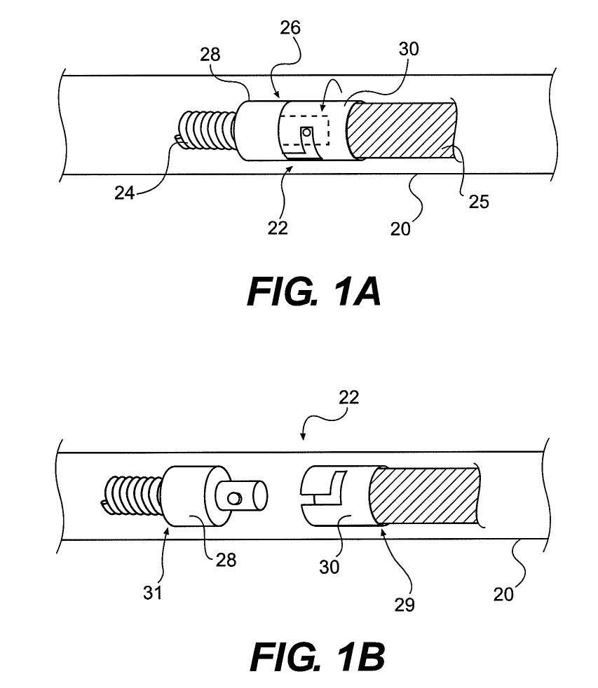

FIGS. 1A and 1B show a portion of catheter 20 cutaway to illustrate a system 22 for delivering an implantable device, in this case an embolic coil 24 (the terms “coil” and “embolic coil” are used interchangeably herein). The system includes a detachable link 26, for releasably joining the embolic coil 24 and a pusher wire 25, the detachable link comprises a first interlocking member 28 and a second interlocking member 30. In one embodiment, the interlocking members 28, 30 have mating surfaces adapted to detach from one another when activated by a user.

One skilled in the art will appreciate that the embolic coil 24 and pusher wire 25 are merely representative of the environment in which detachable link 26 operates, and that a variety of alternative medical devices could be substituted. For example, the systems described herein could be used to delivery a variety of implantable devices in addition, or as an alternative, to the embolic coil. Similarly, the pusher wire represents the variety of control devices for moving an implantable device through a lumen of a medical instrument. In addition, as disclosed in co-pending application Ser. No. 11/248,033, entitled “Multiple Interlocking Detachable Coils,” filed Oct. 12, 2005, and incorporated by reference in its entirety, a second coil, or other such device, could be linked between pusher wire 25 and coil 24 via additional detachable links. Still further, while coil delivery system 22 is generally described with respect to the detachable link traveling through a catheter, one skilled in the art will appreciate that detachable link 26 may travel through a variety of medical instruments, such as, for example, introducers, and that the methods and devices describe herein are equally applicable to any medical device having a lumen for the delivery of a detachable, implantable device. In particular, the term “catheter” as used herein can refer to the variety of medical devices having an inner lumen adapted for receiving a medical instrument and/or implantable device.

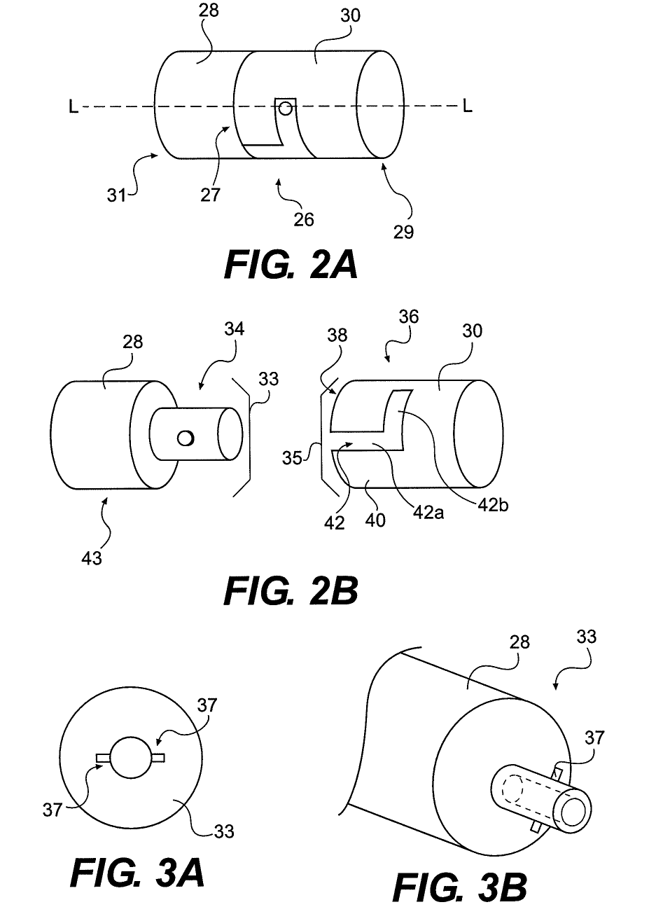

As shown in FIG. 2A, detachable link 26 generally includes a body 27 formed from at least interlocking members 28, 30. Body 27 can have a generally elongate shape extending from a proximal portion 29 to a distal portion 31 along a longitudinal axis L. In one aspect, proximal and distal portions 29, 31 of body 27 can be integrally formed with coil 24 and pusher wire 25 (FIG. 1A and 1B). Alternatively, body 27 can be fixedly mated with the coil and pusher wire. For example, the coil and pusher wire can be welded, adhered, and/or mechanically mated with body 27.

In one aspect, body 27 of detachable link 26 has a cylindrical outer surface that corresponds to an inner lumen of a catheter. However, both catheter and detachable link could have a variety of different shapes including rectangular, oval, D-shaped, triangular, and/or irregular. In addition, the catheter and detachable link could have different shapes from one another.

Body 27, and particularly interlocking members 28, 30, can have a variety shapes and/or sizes that provide a detachable connection that allow detachment at a place and time determined by the user. In one aspect, interlocking members 28, 30 mechanically interlock with one another via first mating surface 33 on first interlocking member 28 and second mating surface 35 on second interlocking member 30.

In one embodiment, first and second mating surfaces have a male/female configuration. First mating surface 33 can be defined by a proximal portion 34 of interlocking member 28. In one aspect, proximal portion 34 has a diameter or cross-sectional width adapted for receipt in the second mating surface. The proximal portion 34 can have a solid configuration with a closed distal end as shown in FIG. 3A. Alternatively, as shown in FIG. 3B proximal portion 34 can be defined by an outer wall with an open inner portion.

First mating surface 33 can have a variety of shapes and sizes which are adapted to mate with second mating surface. For example, first mating surface can be cylindrical, spherical, or similarly shaped to allow the first mating surface to rotate within the second mating surface. Alternatively, first mating surface 33 can have a non-circular cross-sectional shape, including shapes such as rectangular, triangular, oval and/or irregular. FIG. 6, discussed in more detail below, illustrates one exemplary embodiment of a non-cylindrical first mating surface. In another aspect, a first mating surface with a non-circular cross-sectional shape is sized such that the largest cross-sectional width is small enough to allow the first mating surface to rotate within the second mating surface. In still another aspect, the first mating surface can include a longitudinal taper (not illustrated) which facilitates alignment of the first and second mating surfaces.

First mating surface 33 can additionally include surface feature 37, discussed below, for mechanically mating with second mating surface 35. For example, first mating surface can include a protrusion or pin.

Second mating surface 35 includes an opening 38 in a distal portion 36 of interlocking member 30 for receiving at least part of first mating surface 33. Opening 38 can be positioned at the distal most end of interlocking member 30 and allow access into an inner a portion of the second mating surface defined by outer wall 40. In one aspect, the shape of the second mating surface generally conforms to the first mating surface such that whole of the proximal portion 34 is received in the distal portion of interlocking member 30. Alternatively, the first and second mating surfaces could have non-conforming shapes and sizes such that the first mating surface does not completely fill the second mating surface or the second mating surface only covers a portion of the first mating surface.

The outer surface of wall 40 can be sized (e.g., have a diameter) and shaped to match a distal portion 43 of interlocking member 28 such that when mating surfaces 33, 35 are engaged, detachable link 26 has a generally uniform width. For example, the outer surface of wall 40 can have a cylindrical configuration of the same outer diameter and shape as a distal portion 43 of interlocking member 28.

The inner surface of wall 40 can include surface features 42 for mechanically mating with mating surface 33. In one illustrative embodiment, wall 40 includes a slot or recess 42 adapted to receive a pin 37 extending from mating surface 33 similar to a bayonet lock. Recess 42 can extend into wall 40 and in one aspect, recess 42 extends through wall 40. As shown in FIG. 2B, recess 42 can have a first elongate portion 42a extending longitudinally and a second elongate portion 42b extending along an arc of wall 40 in a direction generally transverse to the first elongate portion. In one embodiment, the transverse portion of recess 42 allows mating surface 33, 35, and detachable link 26 to remain engaged as system 22 is pushed and/or pulled through catheter 20.

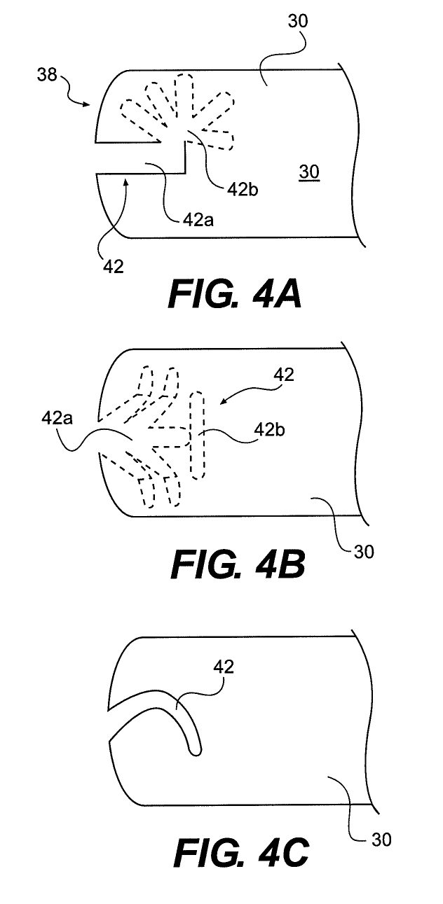

Recess 42 can have a variety of configurations that include at least a portion that extends at an angle with respect to the longitudinal axis of the second interlocking members. FIGS. 4A and 4B illustrate a variety of differently shaped recesses. In FIG. 4A several possible angles for the second portion 42b of the recess are shown in outline. In FIG. 4B several alternative recesses are shown having a first portion 42a extending from opening 38 at an angle with respect to the longitudinal axis of the second interlocking member 30. In yet another embodiment, recess 42 can include a single, continuous portion. For example, FIG. 4C shows recess 42 having a serpentine configuration. Other possible shapes, such as, for example, a spiral or threaded configuration are also contemplated.

Mating surface 35 can include a single or multiple recesses. For example, the second interlocking member could include two, three, or more than three recesses. The multiple recess can correspond to multiple pins 37 on the first mating surface. Alternatively, detachable link 26 could include fewer pins 37 than recesses 42. In use, only some of the recesses would mate with pins.

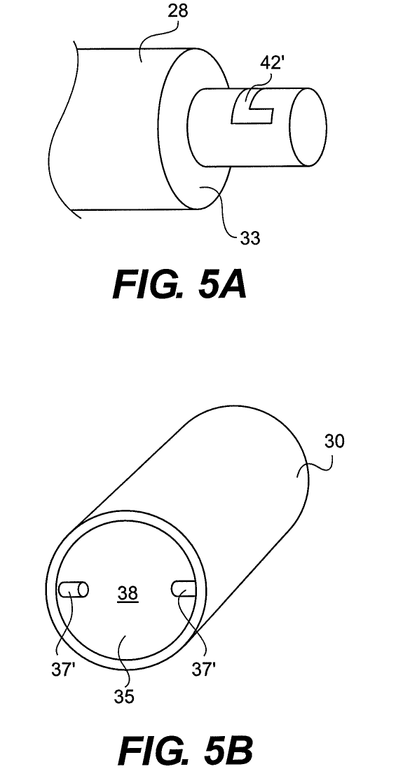

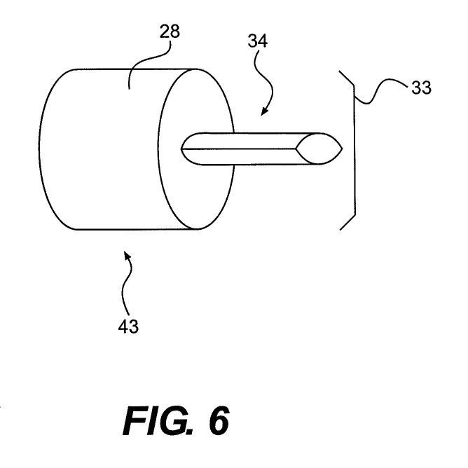

In another embodiment, the male mating surface can include a recess or recesses and the female mating surface can include a pin or pins. FIGS. 5A and 5B show first interlocking member 28 having a recess 42′ positioned on the first mating surface 33′ and corresponding to a pin 37′ on the female mating surface 35′. First and second mating surfaces 33′, 35′ can include the various features and embodiments of mating surfaces 33, 35 discussed above.

Moreover, while male mating surface 33 and female mating surface 35 are illustrated as mated with coil 24 and pusher wire 25, respectively, the first and second mating surfaces could be swapped. For example, first mating surface 33 could be associated with the pusher wire and the second mating surface 35 could be associated with the coil.

The male and female mating surfaces discussed above can allow detachable link 26 to remain engaged as system 22 travels through an inner lumen of catheter 20. Once the detachable link is in a position in which a user wishes to detach coil 24, the first and second interlocking members can be moved relative to one another to allow delivery of coil 24. A user can rotate the pusher wire to rotate second mating surface 35 relative to first mating surface 33 and then move the pusher wire proximally to fully disengage interlocking members 28, 30.

The amount of relative rotation between the mating surfaces necessary to detach the detachable link will depend to the configuration of the first and second mating surfaces. For example, the greater the distance that recess 42, 42′ extends around the circumference of the interlocking member, the more rotation that will be necessary. In one aspect, the detachable link can be detached with less than about two revolutions of the second interlocking member with respect to the first interlocking member. The amount of relative rotation between the first and second interlocking members required to disengage the coil can alternatively be less then about 360 degrees or less than about 180 degrees. In one embodiment, the amount of relative rotation required is in the range of about 5 and 180 degrees, preferably between about 15 and 90 degrees.

In order to achieve disengagement between interlocking members 28, 30, mating surfaces 33, 35 need to move relative to one another. As such, when pusher wire 25 is actuated (e.g., rotated) it is preferable that pusher wire 24 and interlocking member 30 rotate relative to coil 24 and interlocking member 28. In one aspect, friction between the catheter and coil 24 and/or interlocking member 28 is greater than the friction between mating surfaces 33, 35. When pusher wire 25 is rotated, mating surface 35 will rotate (at least to some degree) relative to mating surface 33 due to the frictional resistance cause by contact of coil 24 and/or interlocking member 28 with catheter 20. In other words, rotating pusher wire will cause interlocking member 30 to rotate more than interlocking member 28 because friction will inhibit rotation of interlocking member 28.

In one embodiment, to facilitate detachment, the first and second mating surfaces are sized and shaped to provide minimal frictional engagement. For example, the female mating surface can have a larger size than the male mating surface to allow entry of mating surface 33 into mating surface 35 with a minimal amount of frictional force. Similarly, recess 42, 42′ can have a larger width than the pin to allow the pin to freely slide within the recess. As a result, the friction which must be overcome to detach mating surfaces 33, 35 can be less than the friction required to rotated coil 24 and interlocking member 28. For example, as the user rotates pusher wire 25 and second interlocking member 30, the second mating surface will rotate relative to the first mating surface because the low amount of friction between the mating surfaces is insufficient to transmit enough rotational force to cause interlocking member 28 and coil 24 to rotate.

In another aspect, the outer surfaces of at least one element of system 22 and/or catheter 20 can be adapted to increase or reduce friction. For example, a distal portion of the inner surface of catheter 20 could have a higher coefficient of friction (relative to a more proximal portion of the inner surface of catheter 20) such that when coil 24 and/or interlocking member 28 are positioned in the distal portion of catheter 20, the distal portion of the catheter inhibits movement (e.g., rotation and/or longitudinal movement) of coil 24 and/or interlocking member 28.

In another embodiment, the shape of interlocking member 28 and/or catheter 20 could be configured to facilitate detachment of detachable link 26. For example, instead of relying on a difference in friction (or in addition to differences in friction) a distal portion of catheter could have a profile adapted to grip interlocking member 28. In one such embodiment, a distal portion of catheter 20 could have a smaller inner diameter relative to a more proximal portion of catheter 20. In addition, or alternatively, the distal portion of catheter 20 can include a surface feature or surface features to grip interlocking member 28. For example, the inner surface of the catheter and the outer surface of coil 24 and/or interlocking member 28 can engage via a ribs (not illustrated) positioned on the inner surface of the catheter. In another aspect, the inner surface of catheter 20 can include a protrusion (not illustrated) that mates with a recess (not illustrated) on interlocking member 28 that facilitates relative rotation of interlocking member 28, 30.

In another embodiment, the first and second interlocking members can have different cross-sectional shapes. The shape of the outer surface of interlocking member 28 can be configured to grip (at least a portion of) the inner surface of the catheter while the outer surface of interlocking member 30 can be configured to rotate within catheter. In one aspect, interlocking member 28 can have a shape that corresponds to a distal portion of catheter 20. For example, interlocking member 28 can have an oval shape while interlocking member 30 has a circular cross-sectional shape.

In another embodiment, system 22 can be configured to provide tactile feedback to the user when interlock 26 and/or coil 24 reach a distal portion of catheter 20. For example, the surface features discussed above (e.g., ribs, catheter/interlocking member shape, and/or differences in friction) can cause an increase in force required to move pusher wire 25. The change in force can alert a user, via tactile feedback, that the interlock has reached a position where the coil can be detached. Alternatively, or additionally, the user could rely on a visualization technique to determine the location of interlocking member within catheter 20.

In one embodiment, the pusher wire can be constructed of materials which assist with the transmission of torque between the user and interlocking member 30. Such torquable materials can provide a pusher wire that rotates at its distal end at about the same rate as a user rotates the proximal end. In one aspect, the ratio of rotation of the guide wire at its proximal end relative to its distal ends is less than about 2:1, such that every two rotations of the proximal end result in at least one rotation of the distal end of the pusher wire. In another aspect, the ratio is about 1 to 1. One skilled in the art will appreciate the pusher wire 25 can be constructed from a variety of torqueable materials.

While the first and second mating surface are generally described as allowing the transmission of longitudinal forces in two directions (proximal and distal with respect to the catheter), in another aspect, the detachable link permits only transmission of distal force. For example, FIG. 6 illustrates first mating surface 33 having proximal portion 34 shaped to engage the second mating surface and permit rotational movement of the embolic coil and/or distal movement of the embolic coil. However, in one aspect, first mating surface 33 does not permit withdrawal, or proximal movement, of the coil. For example, the first mating surface does not require a surface feature adapted to mate with the second mating surface. To detach the detachable link, a user moves the first mating surface proximally, allowing the proximal surface of the first mating surface to withdraw from the second mating surface.

Further provided herein is a method for delivering a detachable implantable device. In one embodiment the above described system is used to deliver an embolic coil to a target destination and then detach the coil. The embolic coil can first be moved from an introducer to a catheter by actuating the pusher wire. The detachable link allow a user to control coil movement while inhibiting accidental detachment of the coil caused by variations in lumen diameter sometime found at an interface between an introducer and a delivery catheter.

Once the coil is positioned in the catheter, the user can move the coil along the inner lumen of the catheter until the coil is proximate to the distal end of the catheter. At this point, the user may wish to determine the location of detachable link 26 and coil 24 within the catheter and/or relative to an anatomical feature. The delivery method can include the step of visualizing the relative location of the detachable link and the distal end of the catheter. For example, an imaging technique, such as x-ray, MRI, CT, PET, SPECT and combinations thereof, can be used to visualize the coil and/or detachable link. In addition, or alternatively, as mentioned above, the system 22 can be adapted to provide the user with tactile feedback once the coil and/or detachable link reaches a distal portion of the catheter.

Once system 22 is position in the desired location, the user can actuate the pusher wire to detach the coil. For example, depending on the configuration of the mating surfaces (e.g., pin 37 and recess 42), the user can rotate the pusher wire and/or move the pusher wire longitudinally. In one aspect, the user first rotates the pusher wire to rotate the mating surface 35 relative to mating surface 33 and then moves the pusher wire proximally.

Depending on the location chosen to detach the embolic coil, the coil could still be positioned within the catheter after detachment. In this case, the user can move the pusher wire distally to push the detached coil out of the distal end of the catheter. Since the mating surface (e.g., female mating surface 35) on the detachable link connected to the pusher wire is generally enclosed, system 22 can reduce the chance of interlocking member 30 snaring an implanted coil. This provide a benefit over some conventional systems that have exposed mating features.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An implant delivery system, comprising:
an implantable medical device mated to a pusher wire via a detachable link comprising first and second interlocking members, the first interlocking member including a male mating surface and the second interlocking member including a female mating surface, the first and second body members are adapted to mechanically engage via a pin and a slot for receiving the pin.

2. The system of claim 1, wherein the slot includes a first elongate portion and a second elongate portion extending an at angle with respect to the first elongate portion.

3. The system of claim 1, wherein the pin is positioned on the male mating surface and the slot is positioned on the female mating surface.

4. The system of claim 1, wherein the female mating surface includes a wall defining an internal area.

5. The system of claim 4, wherein the slot extends through the wall.

6. The system of claim 1, wherein the slot extends transversely around a portion of a circumference of the female mating surface.

7. The system of claim 1, wherein the slot has a width greater than a width of the pin to allow the pin to freely slide in the slot.

8. The system of claim 1, wherein the first and second body members mechanically interlock without frictionally mating.

9. The system of claim 1, wherein the male mating surface is closed ended.

10. The system of claim 1, wherein the female mating surface includes an open distal end, a recessed area, and a closed proximal end.

11. The system of claim 1, further comprising multiple pins and multiple slots.

12. The system of claim 1, further comprising a catheter and wherein an outer surface of the detachable link has a shape corresponding to the catheter.

13. An implant delivery system, comprising:
an implantable medical device mated to a pusher wire via a detachable link comprising an elongate body member including first and second interlocking members,
the first interlocking member including an outer surface having at least one protrusion,
the second interlocking member including an wall defining a receiving area for receiving at least a portion of the first interlocking member, the wall further including a recess adapted to receive the protrusion when the first and second interlocking members are engaged.

14. The system of claim 13, wherein the recess includes a first portion that extends along a circumference of the wall.

15. The system of claim 14, wherein the recess includes a second portion extending along a longitudinal axis.

16. The system of claim 13, further comprising multiple protrusions and multiple recesses.

17. A method for delivering a detachable implant, the method comprising the steps of:
providing an embolic coil mated to a pusher wire via a detachable link comprising first and second interlocking members, the first interlocking member including an outer surface having at least one protrusion, and the second interlocking member including an wall defining a receiving area for receiving at least a portion of the first interlocking member, the wall further including a slot for receiving the protrusion;
actuating the pusher wire to move the embolic coil through a catheter; and
rotating the pusher wire to detach the pusher wire from the embolic coil.

18. The method of claim 17, wherein the slot includes a first portion and a second portion extending at an angle with respect to the first portion.

19. The method of claim 17, wherein the step of rotating results in the first interlocking member rotating relative to the second interlocking member.

20. The method of claim 19, wherein the force required to rotate the embolic coil and the first interlocking member is greater than the force required to rotate the first interlocking member relative to the second interlocking member.

21. The method of claim 17, wherein a distal portion of the catheter includes a cross-sectional shape adapted to facilitate detaching the embolic coil.

22. The method of claim 21, wherein the distal portion of the catheter has at least one cross-sectional width that is smaller than a cross-sectional width of the first interlocking member.

23. The method of claim 21, wherein the distal portion of the catheter has surface features adapted to grip one of first interlocking member.

24. The method of claim 21, wherein the first and second interlocking member include different cross-sectional shapes and the cross sectional shape of the first interlocking member corresponds to the distal portion of the catheter.

25. The method of claim 17, further comprising determining the location of the detachable link within the catheter.

26. The method of claim 25, wherein the step of determining the location include using tactile feedback and/or a visualization technique.

27. The method of claim 17, wherein the first and second interlocking members are adapted to mechanically engage without frictionally mating.

28. The method of claim 17, wherein rotating a proximal end of the pusher wire results in a distal end of the pusher wire rotating at least half the number of turns.

29. The method of claim 17, further comprising the step of moving the embolic coil from an introducer into a delivery catheter.

30. A system for delivering an implantable device, comprising:
a medical device having an inner lumen;
an implantable medical device mated to a pusher wire via a detachable link comprising first and second interlocking members, the first interlocking member including a male mating surface and the second interlocking member including a female mating surface, the first and second body members are adapted to mechanically engage via a pin and a slot for receiving the pin,
wherein a distal portion of the inner lumen includes a surface feature adapted to grip at least a portion of the detachable link.

31. The system of claim 30, wherein the surface feature includes a shape corresponding to at least a portion of an outer surface of the first interlocking member.

32. The system of claim 30, wherein the surface feature provides increased friction between the first interlocking member and the inner lumen.
